(19) United States
(12) Patent Application Publication
(10) Pub. No.: US 2011/0263446 A1
(43) Pub. Date: Oct. 27, 2011

(54) KIT USEFUL FOR DETECTING, SEPARATING AND/OR CHARACTERIZING A MOLECULE OF INTEREST

(75) Inventors: Alexandra Le Foll, Deville les Rouen (FR); Vincent Levacher, Fontaine-Sous-Preaux (FR); Celine Perollier, Rouen (FR); Sami Bayoudh, Mont St Aignan (FR); Kaynoush Naraghi, Mont St Aignan (FR)

(73) Assignee: POLYINTELL, Val de Reuil (FR)

(21) Appl. No.: 13/063,041

(22) PCT Filed: Sep. 9, 2009
(86) PCT No.: PCT/IB09/53945

§ 371 Date: Jul. 11, 2011

(30) Foreign Application Priority Data
Sep. 11, 2008 (FR) 0856130

(51) Int. Cl. C40B 30/04; C07K 1/14; G01N 33/53

(52) U.S. Cl. 506/9; 436/501; 530/344

(57) ABSTRACT

A kit for separating and/or characterizing at least one molecule A of interest, the kit including: (i) a compound having the general formula: B—(R)ₙ—Z in which: B represents a hydrogen atom or a detectable labeling entity; R represents a C₁-C₁₀₀₀₀ hydrocarbon unit which may be polymeric or non-polymeric and optionally incorporates one or more heteroatoms, chosen from N, O, S, Br, Cl, F, P, B, Si and/or one or more metals; n represents 0 or 1, with n being equal to 1 when B represents a hydrogen atom; and Z represents a functional group capable of reacting in a click chemistry reaction in order to form a linking function L_{click}; and (ii) a molecularly imprinted polymer dedicated to the molecular recognition of at least said linking function L_{click}.

The present invention relates to the field of molecularly imprinted polymers or MIPs, which are useful for recognizing molecules of interest.

The invention relates more specifically to a kit and a process based on the use of this kit, which prove to be most particularly suitable for the separation, detection and/or characterization of at least one molecule of interest, or of a family of molecules of interest, with increased selectivity.

Industrial techniques and/or processes nowadays require analytical and separation methods and/or tools that are increasingly efficient in terms of selectivity, speed of execution and reliability, especially for characterizing, within a complex medium, the presence of a molecule of interest and/or for isolating it from this medium.

These methods and/or tools are especially useful in the field of organic synthesis, for the recovery of catalysts (for example organometallic catalysts), or for the removal of unwanted by-products, and in particular in biology for diagnosis (for example identification and quantification of biomarkers especially in proteomics, genomics and metabolomics) and in radiopharmacy (purification of radiotracers).

The techniques currently available for performing this type of characterization and/or isolation are mainly solid-phase or liquid-phase chromatography and extraction techniques. These techniques are very often advantageously coupled with labeling of the molecule of interest, the marker then efficiently participating in the isolation of the molecule of interest via one of these extraction techniques, especially in solid phase.

Thus, purification techniques based on preliminary labeling of the molecules of interest using a tag, followed by molecular recognition of said tag have already been described in the literature (Jun-ichi YoShida et al., Chem. Rev. 2002, 102, 3693-3716).

A wide variety of tags has thus been developed in the context of separation techniques based on affinities such as interactions with fluorine, hydrophobic interactions, interactions of hydrogen bonding type, ionic interactions (crown ether-ammonium), metallic chelations, or combinations of these interactions.

Fukase et al. (Synlett 2001, No. 5, 590-596) thus already describe a protocol for separating compounds of interest bearing a unit derived from barbituric acid as tag. This protocol is based on the interaction, via hydrogen bonding, between this barbituric acid derivative and a bis(2,6-diaminopyridine) isophthalic acid amide grafted onto a polystyrene.

However, the use of such a protocol is limited given, in particular, the relative low affinity between barbituric acid and its receptor.

Similarly, Rutjes et al. (Org. Lett., Vol. 8, No. 15, 2006) have performed the selective recycling of catalysts comprising a particular tag, by extraction onto a resin functionalized so as to display complementary interactions with said tag.

In addition, homodimerization of the resin, which is detrimental to the stability of the separation support and thus to its performance qualities, and also certain solubility problems, have also been observed.

Introducing the tag at the end of the reaction, as reported by Jun-ichi Yoshida et al., Chem. Rev. 2002, 102, 3693-3716, however has the drawback of not allowing the separation of the tag that may be present in the medium in a free form, from the tagged compound of interest. Specifically, these two species bear the same tag unit, and consequently have comparable affinity with regard to the separation support.

An alternative to these tags is MIPs.

Specifically, MIPs are advantageous on account of their high specificity and their excellent chemical, mechanical and thermal stability. They are also known for being easily and inexpensively synthesized if the template species is available at low cost, Thus, as reported in Pichon V., Journal of Chromatography A, 2007, 1152, 41-53, the use of MIPs in a solid-phase extraction process thus makes it advantageously possible, in a single step, to obtain an extract enriched in a given molecule of interest.

However, these techniques have the drawback of requiring the synthesis of MIPs that are especially suited to the molecular recognition of each molecule of interest, or of analogs, intended to be separated out.

In general, there is thus a need for a universal separation protocol that is easy to implement, rapid, easy to automate, efficient and/or inexpensive, and which is suited to the extraction of a wide variety of molecules of interest.

There is also still a need for a kit that is useful for the selective isolation of molecules of interest, for example reaction products, reagents and/or catalysts, and/or which allows the characterization of molecules of interest free of readily detectable groups, for example such as chromophores or fluorophores.

The present invention is specifically directed toward satisfying these needs.

Thus, according to one of its aspects, the present invention relates to a kit that is useful for the separation and/or characterization of at least one molecule of interest A, comprising at least:

(i) one compound of general formula (I) below:

B—(R)ₙ—Z (I)

in which:
B represents a hydrogen atom or a detectable labeling species;
R represents a C₁-C_{10 000} hydrocarbon-based unit that may be alternatively polymeric or nonpolymeric and that may optionally incorporate one or more heteroatoms chosen from N, O, S, Br, Cl, F, P, B, Si and/or one or more metals;
n represents 0 or 1, with n being equal to 1 when B represents a hydrogen atom;
Z represents a functional group that is capable of reacting in a click-chemistry reaction, to form a bonding function L_{click}, and

(ii) a molecularly imprinted polymer intended for the molecular recognition of at least said bonding function L_{click}.

When it is a nonpolymer unit, the hydrocarbon-based unit may especially be of C₁₋₁₀₀ and particularly C₁-C₅. It may be linear, branched and/or cyclic, saturated or unsaturated and optionally substituted.

When it is a polymer unit, the hydrocarbon-based unit may then be formed from a repetition of monomer units, especially of C₁-C₁₀. The polymer unit may comprise, for example, from 2 to 1000, especially from 10 to 500 and more particularly from 13 to 150 monomer units. Among the polymer units that are suitable for use in the invention, mention may be made especially of polyethylene glycol, polymethacrylate and polyacrylate.

The kit in accordance with the invention makes it possible to perform quick and easy separation, detection and/or characterization and, in general, faster separation than via standard methods, especially precipitation or chromatography.

It is also suited to use in organic synthesis, for example in multistep synthesis or in combinatorial chemistry, especially via the use of chemically stable species B.

The kit in accordance with the invention may also be used for labeling a reagent and/or a substrate and/or a reaction product and may thus make it possible, for example, to monitor the progress of a reaction via usual detection methods.

To this end, the labeling may be performed, without preference, before, during or at the end of said reaction.

Preferably, the labeling may be performed at the end of the reaction. Specifically, in this case, the labeling will not be liable to interfere with the reaction conditions.

In the context of the present invention, the term “molecule of interest” is intended to denote any species that it is desired to separate from a medium and/or to characterize.

This may be any type of species comprising, naturally or after modification, at least one functional group W.

The modification of a species in order to make it bear a functional group W may be performed according to any method conventionally used in organic chemistry. These methods are considered as forming part of the general knowledge of a person skilled in the art.

For the purposes of the present invention, the term “C₁-Cₓ hydrocarbon-based unit” means, in the context of the present invention, a hydrocarbon-based unit comprising a total number of carbon atoms between 1 and x.

In the context of the present invention, the term “monomer unit” means the smallest constituent unit whose repetition leads to a macromolecule or a polymer unit.

The kit in accordance with the invention is intended to be used for the separation, detection and/or characterization of at least one molecule of interest A that has been pregrafted with a compound of general formula (II) below:

X—R′—Y (II)

in which:
X represents a functional group that is reactive toward at least one functional group W borne by said molecule of interest A;
R′ represents a C₁-C_{10 000} hydrocarbon-based unit that may be alternatively polymeric or nonpolymeric and that may optionally incorporate one or more heteroatoms chosen from N, O, S, Br, Cl, F, P, B, Si and/or one or more metals; and
Y represents the functional group that is reactive in a click-chemistry reaction toward the functional group Z mentioned previously, to form the bonding function r.

When it is a nonpolymer unit, the hydrocarbon-based unit may especially be of C₁₋₁₀₀ and particularly C₁-C₅. It may be linear, branched and/or cyclic, saturated or unsaturated and optionally substituted.

When it is a polymer unit, the hydrocarbon-based unit may then be formed from a repetition of monomer units, especially of C₁-C₁₀. The polymer unit may comprise, for example, from 2 to 1000, especially from 10 to 500 and more particularly from 13 to 150 monomer units. Among the polymer units that are suitable for use in the invention, mention may be made especially of polyethylene glycol, polymethacrylate and polyacrylate.

This pregrafting step may be performed according to any method conventionally used in organic chemistry. These methods are also considered as forming part of the general knowledge of a person skilled in the art.

According to one embodiment, the kit in accordance with the invention may thus also comprise at least one compound of general formula (II) as defined previously.

The kit in accordance with the invention may especially comprise at least two compounds of general formula (II) as defined previously, said compounds comprising, respectively, identical functional groups Y and functional groups X of different nature.

An object of the present invention is thus to provide users with a separation, detection and/or characterization kit that is suited to a wide range of molecules of interest, and that is modulable both as regards its design and as regards its use, as emerges clearly on reading the text hereinbelow.

In particular, it is possible according to the invention to modify the kit via the choice of the compound of general formula (I), and more particularly via the nature of the functional group Z and/or the nature of R and/or the nature of B.

It is also possible according to the invention to modify the kit via the nature of the molecularly imprinted polymer.

Thus, according to one embodiment, the molecularly imprinted polymer may be intended for the molecular recognition of a unit R-L_{click}, B-L_{click}, B—R-L_{click} or even R-L_{click}-R′, B-L_{click}-R′ or B—R-L_{click}-R′.

It is also possible to modify the kit via the choice of the compound(s) of general formula (II) that may be present, and in particular via the nature of the functional groups X and/or Y.

These various choices, and especially the choice of the functional groups X, Y and Z, afford a modulable kit, suited to the detection, separation and/or characterization of a wide variety of molecules of interest.

Moreover, the various choices concerning the nature of B also make it possible to provide a kit that is especially modulable in terms of detection and characterization techniques and/or in terms of sensitivity.

According to another of its aspects, a subject of the invention is also a process for separating, detecting and/or characterizing at least one molecule of interest A that may be present in a medium, and in particular in a complex medium, characterized in that it comprises at least one step of using a kit in accordance with the invention.

According to another of its aspects, the invention also relates to the use of a kit in accordance with the invention for the purposes of extraction, detection, separation, purification, absorption, adsorption, retention or controlled release, qualification and/or quantification of biomarkers in biology (proteomics, metabolomics or genomics) or alternatively in applications chosen from sensors, catalysis of chemical reactions, radiopharmacy, parallel synthesis, screening of molecules, directed chemical synthesis, sample treatment, combinatorial chemistry, chiral separation, group protection, equilibrium shifting, medicaments using polymers, and encapsulation.

As indicated previously, the molecule of interest may especially be present in a complex medium.

In the context of the present invention, the term “complex medium” is intended to denote a medium comprising, besides the molecule(s) of interest, at least one or more other associated species.

By way of example of complex medium according to the invention, mention may be made especially of reaction media derived from chemical synthesis, but also bodily fluids such as blood, plasma, saliva, urine, bile, tears, maternal milk, or alternatively culture media, cell lyzates, plant extracts, foods, environmental media (soil, water or air), drinks such as wine, milk, fruit juices or beer, or alternatively gaseous media.

The complex medium is preferably a reaction medium comprising one or more reaction products and, optionally, at least one reagent in excess and/or a substrate in excess and/or unwanted reaction by-products and/or a catalyst.

According to one embodiment, the kit in accordance with the invention may thus be useful for the separation or detection of a molecule of interest present in a complex medium, for example a catalyst, a substrate, a reagent, a product or a by-product present in a reaction medium.

In the context of the present invention, the term “functional group” is intended to denote a group of atoms forming a reactive function.

This may be any reactive function known to those skilled in the art, and especially an azide, alkyne, nitrile, carboxylic acid, ester, anhydride, acid halide, amide, iso(thio)cyanate, epoxide, thiol, amine, aziridine, ketone, aldehyde, diene, alkene or hydroXyl function.

The term “bonding function” is intended to denote a chemical function whose formation makes it possible to covalently connect two initially separate chemical species.

For example, a bonding function L may be derived, according to the invention, from the reaction of a functional group X onto a complementary functional group W, and make it possible to covalently connect a compound of general formula (II) and a molecule of interest A.

Similarly, a bonding function L_{click} may be derived, according to the invention, from the click-chemistry reaction between a functional group Y and a complementary functional group Z, and make it possible to covalently connect a compound of general formula (I) and a compound of general formula (II).

Bonding functions that may especially be mentioned include triazole, tetrazole, ester, amide, urethane, cyclohexene, carbamate, silyl ether and imine functions.

The bonding functions under consideration according to the present invention may or may not be cleavable.

The term “cleavable bonding function” is intended to denote a covalent bond that can be broken under relatively mild and/or selective conditions. A cleavable bonding function may be selectively broken under conditions such that the breaking of the other covalent bonds is avoided.

A review of the various cleavable bonding functions known to those skilled in the art is espetially detailed in the publication Guillier at al. (Chem. Rev., 2000, 100, 2091-2157).

For example, a disulfide bond —SS— may be broken in the presence of a thiol or by selective irradiation with electromagnetic radiation at a specific wavelength, without resulting in breaking of the other bonds, such as the carbon-carbon, carbon-oxygen, carbon-sulfur or carbon-nitrogen bonds, which may also be present, or alternatively in the presence of a reducing agent such as a phosphine.

Cleavable bonding functions that may also be mentioned include ester, amidine, silyl ether, silyl-R (R=O, N, S or C), carbamate, imine and enamine bonds.

Detection, Separation and/or Characterization Kit

The separation, detection and/or characterization kit according to the invention comprises at least one compound of general formula (I) and at least one molecularly imprinted polymer as defined previously.

It may also comprise at least one, and preferably at least two, compounds of general formula (II) as defined previously.

For the purposes of the invention, the term “kit” is intended to denote a packaging assembly in which said compound(s) of general formula (I), molecularly imprinted polymer(s) and optionally compound(s) of general formula (II) are packaged separately from each other, for example in separate compartments or on separate supports.

According to one embodiment, the kit according to the invention may comprise various types of compound of general formula (I) and/or of molecularly imprinted polymer and/or optionally of compound of general formula (II) as a function especially of the target molecule(s) of interest.

In particular, the nature of the compound of general formula (II), and especially that of its functional group X, may be adjusted according to the target molecule of interest, and more particularly according to the nature of the functional group W borne by said molecule of interest.

Similarly, the nature of the compound of general formula (I), and especially that of its functional group Z, may be chosen as a function of the functional group Y grafted onto said molecule of interest.

### Compound of General Formula (I)

As indicated previously, the separation, detection and/or characterization kit according to the invention comprises at least one compound of general formula (I) below:

B—(R)ₙ—Z (I)

in which:
B represents a hydrogen atom or a detectable labeling species;
R represents a C₁-C_{10 000} hydrocarbon-based unit that may be alternatively polymeric or nonpolymeric and that may optionally incorporate one or more heteroatoms chosen from N, O, S, Br, Cl, F, P, B, Si and/or one or more metals;
n represents 0 or 1, with n being equal to 1 when B represents a hydrogen atom; and
Z represents a functional group capable of reacting in a click-chemistry reaction to form a bonding function L_{c1ick}.

When it is a nonpolymer unit, the hydrocarbon-based unit may especially be of C₁₋₁₀₀ and particularly C₁-C₅. It may be linear, branched and/or cyclic, saturated or unsaturated and optionally substituted.

When it is a polymer unit, the hydrocarbon-based unit may then be formed from a repetition of monomer units, especially of C₁-C₁₀. The polymer unit may comprise, for example, from 2 to 1000, especially from 10 to 50 and more particularly from 13 to 150 monomer units. Among the polymer units that are suitable for use in the invention, mention may be made especially of polyethylene glycol, polymethacrylate and polyacrylate.

According to one embodiment of the invention, and in particular when the kit in accordance with the invention is intended for the characterization of at least one molecule of interest, B may represent a detectable labeling species.

It may be any type of species that is detectable by visible colorimetry, for example with the naked eye, by radiochemistry, by nuclear medicine, for instance by scintigraphy, by imaging, by resonance (MRI), by X-ray, by light scattering, by mass spectrometry, by spectroscopy, for instance by UV-visible fluorescence, by infrared spectroscopy, by surface-plasmon resonance spectroscopy, by chemiluminescence, by interference and refraction spectroscopy, by Raman scattering, by ultrasonication, by radioactivity, by refractometry, by optical, piezoelectric, magnetic or acoustic detection, by electrochemistry, by conductivity, by pH-metry, or biologically, and preferably with the naked eye.

Examples of detectable labeling species that may be mentioned include dyes, fluorophores and chromophores.

Such a species may be, for example, a compound chosen from aromatic derivatives, coumarin derivatives and azo derivatives.

For the purposes of the invention, the term “aromatic derivative” is also intended to cover heteroaromatic derivatives, i.e. aromatic derivatives comprising at least one aromatic heterocycle, said heterocycle comprising at least one heteroatom chosen, for example, from N, O and S.

According to one particular embodiment, 7 is chosen from azides, alkynes, nitriles, dienes and alkenes.

According to one particular embodiment of the invention, B may be a labeling species that is detectable, for example by fluorescence, only after the formation of the bonding function L_{click}.

It may especially be a species that is capable of generating a detectable signal during the click-chemistry reaction.

Examples of such species are especially described in patent application WO 2005/103 705.

According to one preferred embodiment of the invention, the separation, detection and/or characterization kit in accordance with the invention comprises at least one compound of general formula:

B—(R)ₙ—Z (I)

in which;
B represents a labeling species chosen from dyes, fluorophores and chromophores, in particular a heteroaromatic derivative, a phenyl derivative or a coumarin derivative, and even more particularly a phenyl derivative;
R represents a C₁-C_{10 000} hydrocarbon-based unit that may be alternatively polymeric or nonpolymeric and that may optionally incorporate one or more heteroatoms chosen from N, O, S, Br, Cl, F, P, B, Si and/or one or more metals; and in particular a unit —CH₂—;
n represents 0 or 1, with n being equal to 1 when B represents a hydrogen atom; and
Z represents a functional group chosen from azides, alkynes, nitriles, dienes, and alkenes, and in particular an azide.

When it is a nonpolymer unit, the hydrocarbon-based unit may especially be of C₁₋₁₀₀ and particularly C₁-C₅. It may be linear, branched and/or cyclic, saturated or unsaturated and optionally substituted.

When it is a polymer unit, the hydrocarbon-based unit may then be formed from a repetition of monomer units, especially of C₁-C₁₀. The polymer unit may comprise, for example, from 2 to 1000, especially from 10 to 500 and more particularly from 13 to 150 monomer units. Among the polymer units that are suitable for use in the invention, mention may be made especially of polyethylene glycol, polymethacrylate and polyacrylate.

According to one embodiment, it is a compound of general formula (I) in which B represents a phenyl derivative, n is equal to 1 and R represents a unit —CH₂—, and Z represents an azide.

According to another embodiment, it is a compound of general formula (I) in which B represents a coumarin derivative, n is equal to 0 and Z represents an azide.

According to another embodiment, it is a compound of general formula (I) in which B represents a phenyl derivative, n is equal to 1 and R represents a unit —CH₂NHCO—, and Z represents an alkyne.

### Compound of General Formula (II)

As indicated previously, the separation, detection and/or characterization kit according to the invention may also comprise at least one and preferably at least two compound(s) of general formula (II) below:

X—R′—Y (II)

in which:
X represents a functional group that is reactive toward at least one functional group W borne by said molecule of interest A;
R′ represents a C₁-C_{10 000} hydrocarbon-based unit that may be alternatively polymeric or nonpolymeric and that may optionally incorporate one or more heteroatoms chosen from N, O, S, Br, Cl, F, P, B, Si and/or one or more metals; and
Y represents the functional group that is reactive in a click-chemistry reaction toward the functional group Z, to form the bonding function L_{click}.

When it is a nonpolymer unit, the hydrocarbon-based unit may especially be of C₁₋₁₀₀ and particularly C₁-C₅. It may be linear, branched and/or cyclic, saturated or unsaturated and optionally substituted.

When it is a polymer unit, the hydrocarbon-based unit may then be formed from a repetition of monomer units, especially of C₁-C₁₀. The polymer unit may comprise, for example, from 2 to 1000, especially 10 to 500 and more particularly 13 to 150 monomer units. Among the polymer units that are suitable for use in the invention, mention may be made especially of polyethylene glycol, polymethacrylate and polyacrylate.

The functional group X may be chosen, for example, from carboxylic acid, amine, hydroxyl, hydroxylamine, nitrile, thiol, anhydride, acid halide and iso(thio)cyanate functions.

According to one embodiment, the functional group X may be capable of forming a bonding function that is optionally cleavable by reaction with said functional group W.

According to another embodiment, the unit R′ may incorporate at least one cleavable bonding function.

According to one preferred embodiment of the invention, the detection, separation and/or characterization kit in accordance with the invention may comprise at least one compound of general formula:

X—R′—Y (II)

which:
X represents a functional group chosen from carboxylic acid, amine, hydroxyl, hydroxylamine, nitrile, thiol, anhydride, acid halide and iso(thio)cyanate functions and in particular chosen from carboxylic acids and hydroxyls;
R′ represents a C₁-C_{10 000} hydrocarbon-based unit that may be alternatively polymeric or nonpolymeric and that may optionally incorporate one or more heteroatoms chosen from N, O, S, Br, Cl, F, P, B, Si and/or one or more metals; and in particular a unit —CH₂—, —CH₂—N(H)—C(O)-Ph-; —CH₂—O—C(O)-Ph or —CH₂—O—C(O) (CH₂)₄—CH₃ and
Y represents a functional group chosen from azides, alkynes, nitriles, dienes and alkenes, and in particular an alkyne.

when it is a nonpolymer unit, the hydrocarbon-based unit may especially be of C₁₋₁₀₀ and particularly C₁-C₅. It may be linear, branched and/or cyclic, saturated or unsaturated and optionally substituted.

When it is a polymer unit, the hydrocarbon-based unit may then be formed from a repetition of monomer units, especially of C₁-C₁₀. The polymer unit May comprise, for example, from 2 to 1000, especially from 10 to 500 and more particularly from 13 to 150 monomer units. Among the polymer units that are suitable for use in the invention, mention may be made especially of polyethylene glycol, polymethacrylate and polyacrylate.

According to one embodiment, it is a compound of general formula (II) in which X represents a carboxylic acid function, R′ represents a unit —CH₂—N(H)—C(O)-Ph- and Y represents an alkyne.

### Click-Chemistry Reaction

As has been fully described in the article by Sharpless K. B. et al. (Angew. Chem. Int. Ed., 2001, 40, 2004-2021), click chemistry corresponds to stereospecific reactions leading to the formation of at least one covalent bond between a carbon atom and a heteroatom under operating conditions that are simple to perform and in which the presence of water or oxygen generally has no influence on the reaction progress. These reactions are occasionally performed without solvent or in the presence of a nonpolluting solvent (such as water) or a solvent that can be easily removed. The desired product is readily isolable and obtained in good yields, without formation of interfering by-products.

The mild operating conditions (for example: reaction at room temperature using water as reaction solvent) and the very high yields of these reactions are very much suited to treating molecules of any type, and especially fragile molecules.

More generally, it is known that this type of reaction also has a driving force of greater than 20 kcal.mol⁻¹. In this type of reaction, the placing in contact of two main substrates, whose functional groups are complementary, leads efficiently to the desired product.

According to one embodiment, the click-chemistry reaction used according to the invention may be:
a cycloaddition reaction of unsaturated species, in particular a 1,3-dipolar cycloaddition reaction or a reaction of Diels-Alder type;
a nucleophilic substitution reaction, in particular a reaction for the opening of strained electrophilic heterocycles such as epoxides, aziridines, aziridinium ions and episulfonium ions,
a reaction on carbonyls, with the exception of aldol chemistry, in particular a reaction for the formation of ureas, thioureas, aromatic heterocycles, oxime ethers, hydrazones or amides;
an addition reaction to carbon-carbon multiple bonds, in particular an epoxidation, dihydroxylation, aziridination or sulfenyl halide addition reaction, or alternatively a Michael-type addition reaction.

According to one embodiment, the functional groups Y and Z are functional groups bearing, respectively, (i) at least one sp² hybrid carbon, preferably a group C═C, or even a conjugated system C═C—C═C, or an sp hybrid carbon, preferably a group C≡C (for example obtained by Sonogashira coupling) or C≡N, and (ii) an azide group, and vice versa.

Among the complementary couples between the functional group Y and the functional group Z, mention may be made especially of:
alkyne or nitrile (group Y)/azide (group Z) couples;
azide (group Y)/alkyne or nitrile (group Z) couples.

A couple that is particularly preferred according to the invention is the azide (group Y)/alkyne (group Z) couple.

The click-chemistry reaction between the functional groups Y and Z is preferably a 1,3-dipolar cycloaddition reaction or a reaction of Diels-Adler type. It may especially be a 1,3-cycloaddition reaction between a dipole, which may be, for example, an azide group N₃, and a dipolarophile, which may be, for example:
either an alkyne group; the reaction leading in this case to a triazole,
or a nitrile group; the reaction leading in this case to a tetrazole, the group Y preferably being, in this case, the dipole.

When the dipolarophile is an alkyne, it may be terminal or substituted with an alkyl group (SiR₃) or MgX (R is a hydrocarbon-based unit and X is a halide), and preferably terminal.

The click-chemistry reaction may optionally be performed in the presence of a catalyst.

In the case of a coupling by 1,3-cycloaddition, and especially Huisgen 1,3-cycloaddition, it is recommended to use a metal catalyst, advantageously a transition metal and particularly Cu^{I}, Ru or Mg.

An MIP may also be used to promote the cycloaddition reaction, as reported by Zhang H. at al. J. Am. Chem. Soc. 2006, 4178, thus avoiding the use of a metal. In other words, according to one particular embodiment, the molecularly imprinted polymer can per se act as the catalyst.

Two sorts of alkynes and nitriles that may be used as functional group Y or Z may specifically be distinguished.

The first sort is formed by dipolarophiles with an electron-accepting group which are said to be “activated” and which react readily with the dipole. These “activated” alkynes or nitriles may occasionally react with the azide virtually quantitatively in the absence of catalyst.

The second sort is formed by “unactivated” alkynes and nitriles for which the cycloaddition reactions, in particular Huisgen 1,3-cycloaddition, using them require prior activation via the action of a catalyst, preferably also with a base. By way of example, it is especially possible to use a copper catalyst Cu^{I} generated in situ, by reaction of a source of Cu^{II}, such as (CuSO₄.5H₂O), and of a base such as sodium ascorbate, which will reduce the Cu^{II} to Cu^{I}. If the solvent is aqueous, this approach is favored. In the case of unactivated alkynes, the addition of a base is recommended to facilitate the loss of the proton from the alkyne and thus to promote the initiation of the reaction. Specifically, the mechanism envisioned for this reaction proceeds via a ring in which the base deprotonates the alkyne and the copper acetylide is then formed.

The acetylide then reacts with the azide. Formation of the new triazole derivative ligand is thus obtained. This new complex then loses Cu^{I}. The catalyst is regenerated and the final product is obtained.

Although click-chemistry reactions, and in particular 1,3-cycloadditions, are relatively insensitive to the reaction solvent, the use of polar solvents may facilitate them. Thus, the click-chemistry reaction is preferably performed in the presence of at least one polar solvent that may be chosen especially from water, alcohols, acetone, acetonitrile and dimethylformamide (DMF), and mixtures thereof.

### Molecularly Imprinted Polymer

The separation, detection and/or characterization kit in accordance with the invention also comprises at least one molecularly imprinted polymer (MIP) for the molecular recognition of at least the bonding function L_{click} under consideration.

It may especially be an MIP comprising at least one recognition site that is capable of interacting with at least said bonding function L_{click}.

For the purposes of the present invention, the following definitions apply:
“recognition site”, a site existing in the cavity of the matrix of the MIP that participates efficiently in the recognition of a species;
“interaction” taking place between the bonding function L_{click} and a recognition site, or alternatively between the unit R-L_{click}, B-L_{click}, B—R-L_{click}, or even R-L_{click}-R′, B-L_{click}-R′ or B—R-L_{click}-R′ and a recognition site, the formation of weak bonds (for example of van der Waals bond, hydrogen bonding, pi donor-pi acceptor bond or hydrophobic interaction type) and/or strong bonds (for example of ionic bond, covalent bond or ionocovalent bond, coordination bond and dative bond type).

Such a molecularly imprinted polymer may be obtained according to any polymerization reaction known to those skilled in the art, and, for example, as indicated hereinbelow.

The polymerization step of the MIP around a template species involves techniques that are known per se to those skilled in the art. Reference may thus be made to the article by Peter A. G. Cormack at al., Journal of Chromatography B, 804 (2004) 173-182, which presents a review of the available techniques as regards aspects of polymerization of MIPs.

More specifically, there are mainly two approaches possible for making MIPs, the covalent approach developed by Wulff in document U.S. Pat. No. 4,127,730 and the noncovalent approach developed by Mosbach in document U.S. Pat. No. 5,110,833. These two approaches may also be combined.

It is thus possible to use the first approach of covalent type for the preparation of the MIP and the second approach to obtain recognition via noncovalent interactions, as is disclosed, for example, in M. J. Whitcombe at al. “A New Method for the Introduction of Recognition Site Functionality into Polymers prepared by molecular Imprinting: Synthesis and Characterization of Polymeric Receptors for Cholesterol” J. Am. Chem. Soc., 1995, 117, 7105-7111.

It is also possible to use the first and second approaches for the preparation of the MIP, and also to obtain recognition via covalent and noncovalent interactions simultaneously for the same target molecule. Thus, the interaction proceeds at least two different sites of the recognition site, as is disclosed, for example, in Wulff G, at al. Macromol. Chem. Phys. 1989, 190, 1717 and 1727.

A third approach (“semicovalent”) consists in using for the synthesis of MIPs molecules that are specific according to the intended target molecule(s), and in particular at least partly monomers derived from a target molecule, thus acting partly as polymer of the matrix and acting partly as template species. In other words, a part of these monomers, once polymerized, is intended to be removed so as to give rise to the recognition sites.

The molecularly imprinted polymers that are suitable for use in the process according to the invention are preferably obtained according to the noncovalent approach.

In general, these MIPs are obtained by copolymerizing monomers and crosslinking agent(s) in the presence of a species whose imprint it is specifically desired to form. The monomers become specifically arranged around this species, also known as the “template species”, via strong or weak interactions, and are then generally polymerized in the presence of a high content of crosslinking agent. After polymerization, the species is extracted from the polymer material and thus leaves its molecular imprint in cavities within the material, which constitute real synthetic receptors comparable to biological receptors of antibody type.

In the context of the present invention, a chemical species comprising at least one L_{click} unit, for example at least one R-L_{click}, B-L_{click} or B—R-L_{click}, or even R-L_{click}-R′, B-L_{click}-R′ or B—R-L_{click}-R′ unit, is preferably used as template species.

The NIP, or more specifically the matrix constituting it, may thus be formed by radical copolymerization. Vinyl monomers, styrene-based monomers and methacrylic acid-based monomers are monomers that are particularly suitable for this technique. Any initiator may be used, such as azobisisobutyronitrile (AIBN).

As monomers that are useful for the synthesis of MIPs, mention may be made of:

acid monomers: methacrylic acid (MAA), p-vinylbenzoic acid, acrylic acid. (AA), itaconic acid, 2-(trifluoromethyl)acrylic acid (TFMAA), acrylamido-(2-methyl)propanesulfonic acid (AMPSA) and 2-carboxyethyl acrylate,

basic monomers: 4-vinylpyridine (4-VP), 2-vinylpyridine (2-VP), 4-(5)-vinylimidazole, 1-vinylimidazole, allylamine, N,N′-diethylaminoethyl methacrylamide (DEAEM), N-(2-aminoethyl)methacrylamide, N,N′-diethyl-4-styrylamidine, N,N,N-trimethyl aminoethyl methacrylate, N-vinylpyrrolidone (NVP) and ethyl ester of urocanic acid,

neutral monomers: acrylamide, methacrylamide, 2-hydroxyethyl methacrylate (2-HEMA), trans-3-(3-pyridyl)acrylic acid, acrylonitrile (AN), methyl methacrylate (MMA), styrene and ethylstyrene.

Crosslinking agents that may especially be mentioned include p-divinylbenzene (DVB), 1,3-diisopropenyl-benzene (DIP), ethylene glycol dimethacrylate (EGDMA), tetramethylene dimethacrylate (TDMA), N,O-bisacryloyl-L-phenylalaminol, 2,6-bisacryloylamidopyridine, 1,4-phenylene diacrylamide, N,N′-1,3-phenylenebis(2-methyl-2-proloenamide) (PDBNP), 3,5-bisacrylamidobenzoic acid, 1,4-diacryloylpiperazine (DAP), N,N′-methylene-bisacrylamide (MDAA), N,N′-ethylenebismethacrylamide, N,N′-tetramethylenebismethacrylamide, N,N′-hexa-methylenebismethacrylamide, anhydroerythritol dimethacrylate, 1,4;3,6-dianhydro-D-sorbitol 2,5-dimethacrylate, isopropoylenebis(1,4-phenylene) dimethacrylate, trimethylpropane trimethacrylate (TRIM), pentaerythritol triacrylate (PETRA) and pentaerythritol tetraacrylate (PETER).

The crosslinking agent is preferably chosen from ethylene glycol dimethacrylate and divinylbenzene.

It falls within the general competence of a person skilled in the art to prepare the MIP in accordance with the invention having the required properties according to the intended application, and especially the required recognition properties toward the bonding function L_{click}.

The synthesis of the molecular imprint may be performed by solution, emulsion or suspension polymerization., by precipitation, in microemulsion, by dispersed-phase polymerization or under conditions for the preparation of microgels.

The matrix of the molecular imprint formed may be of polyacrylate, polymethacrylate, polyacrylamide, polyvinyl, polyacrylein, polyacrylonitrile, polyvinyl alcohol), polyalkyl vinyl ketone, polybenzothiazole, poly(bis-phenol A carbonate), poly(diallyldimethylammonium chloride), polyvinyl chloride, polysiloxane, polyether aromatic, polyether sulfone, polyetherimide, polyethyleneimine, polyimide, polyimidazole, polyoxymethylene, polyoxazole, polyoxyphenylene, polyoxytetramethylene, polyvinyl alkyl ether, polyvinylpyrrolidone or polyvinyl methyl ketone nature.

The MIPs in accordance with the invention, for example obtained according to the preparation process as described previously, advantageously have high molecular recognition for the bonding function L_{click}.

According to one embodiment, the MIPs in accordance with the invention have high molecular recognition for the unit R-L_{click}, B—R-L_{click}, or even R-L_{click}-R′, B-L_{click}-R′ or B—R-L_{click}-R′.

The unit(s) R and/or R′ may especially incorporate at least one auxiliary functional group that is capable of interacting with said molecularly imprinted polymer via interactions identical or similar to those existing between said bonding function L_{click} and said MIP.

By way of example, if the interactions between the bonding function L_{click} and the MIP are of weak bond formation type, especially of hydrogen bonding type, the unit(s) R and/or R′ may incorporate at least one functional group of amide, carboxylic acid or hydroxyl type that is capable of showing this same type of interaction.

The presence of such auxiliary functions advantageously makes it possible to reinforce the interactions and thus to increase the performance qualities and/or the separation selectivity.

The MIPs in accordance with the invention have, on the other hand, poorer molecular recognition for chemical species lacking a bonding function L_{click}, and comprising, for example, only a functional group X or Y in a reactive form, such as, for example, compounds of general formula (I) and (II).

This aspect is especially illustrated by Examples 2 and 4, which demonstrate the large difference in selectivity between these species toward the same MIP in accordance with the invention.

In other words, the MIPs under consideration in the context of the present invention are intended for the recognition of the unit featured by the bonding function L_{click} established between, on the one hand, a molecule of interest A, where appropriate pregrafted with a compound of general formula (II), and, on the other hand, a compound of general formula (I) as defined previously.

It should be noted that this bonding function L_{clic}), is necessarily internal to the structure of a given species, and cannot in particular be located at an end thereof in so far as it results from a reaction between two preexisting species. The MIPs under consideration in the context of the present invention are therefore not intended for the recognition of ends of a species, but, in contrast, for the recognition of a bonding function present within the structure thereof.

Consequently, the MIPs in accordance with the invention are obtained from at least one template species comprising at least the unit featured by this bonding function L_{click}.

Such a template species is different than the molecule of interest A, which lacks such a bonding function L_{click} within the meaning of the invention.

It should be noted that the adduct resulting from the formation of the bonding function L established, on the one hand, between a molecule of interest A, where appropriate pregrafted with a compound of general formula (II), and, on the other hand, a compound of general formula (I), has greater affinity for the recognition sites of the MIP under consideration in the context of the present invention, than related compounds not comprising such a unit featured by the bonding function L_{click} such as, for example, the molecule of interest A, the compound of general formula (I) and the compound of general formula (II).

Given this difference in affinity, the placing in contact of the MIP under consideration in the context of the present invention with a medium comprising both such an adduct and other related compounds lacking units featured by the bonding function L_{click}, especially the starting compounds that have led to this adduct (namely the molecule of interest A, the compound of general formula (II) and, where appropriate, the compound of general formula (I)) will lead to a selective extraction of said adduct by the MIP.

Thus, when the MIP under consideration in the context of the present invention is placed in contact with a medium comprising both such an adduct and such other related compounds, it will comprise in its recognition sites at least 50% of said adduct, preferably at least 60% of said adduct, preferably at least 70% of said adduct, in particular at least 80% of said adduct and preferably at least 90% of said adduct, relative to the total amount of adduct present in said medium.

### Support

The MIPs in accordance with the invention may be used on any suitable support.

For the purposes of the invention, the term “support” is understood very broadly as meaning any solid, flexible or rigid substrate on or in which the MIPs may be attached, bonded, deposited, synthesized in situ, filled and/or packaged.

The supports that may be used according to the invention may be of any nature, for instance of biological, nonbiological, organic or inorganic nature, or alternatively a combination thereof. They may be in any form, and may especially be in the form of particles, gels, sheets, tubes, spheres, capillaries, points, films or wells, of any size and of any shape.

They may be, for example, in the form of particles of uniform size, especially between 10 nm and 10 mm and preferably between 25 and 80 μm, which may subsequently be packaged in cartridge form.

In general, the MIPs may be used, for example, on or in a support chosen from an SPE cartridge, a multiwell plate, for instance a 96-well plate, a patch, a teabag, a microtube, an HPLC column, a strip, chips, slides, silica plates, wafers, a porous surface, a nonporous surface or a microfluidic system.

According to one embodiment of the invention, the molecularly imprinted polymers may be used on an extraction column, for example on an SPE cartridge, optionally graduated.

Thus, according to one embodiment of the invention, the detection, separation and/or characterization process in accordance with the invention as described below may comprise at least one step of solid-phase extraction (SPE).

A solid-phase extraction procedure generally comprises three or four steps. The first is the conditioning of the adsorbent contained in the extraction cartridge, which allows the support to be wetted, solvating the functional groups present at its surface. During the second step, the solution to be treated is percolated through the MIP, so that the species that have no affinity therewith (i.e. the compounds lacking the bonding function L_{click}) are not retained. On the other hand, the species with strong affinity for the adsorbent (compounds comprising at least one bonding function L_{click}) remain on the support after this step.

An additional washing step is performed so as to remove the species weakly retained (compounds lacking the bonding function L_{click}) by the support, by means of a solvent with an eluting force that is suitable to elute these species while retaining the species showing high affinity for the adsorbent (compound comprising at least one bonding function L_{click}) on the support.

Finally, elution of the species showing high affinity for the adsorbent is performed by passing a solvent specifically chosen to allow cleavage of the recognition interactions acting between the species with strong affinity for the adsorbent and the MIP.

At the end of this extraction and release process, a purified solution, optionally enriched in the species with strong affinity for the adsorbent, may thus be obtained.

Typically, the solvents used during a solid phase extraction may be organic solvents, for instance acetonitrile, methanol or dichloromethane, aqueous solvents, for instance water or buffer solutions, the solvents possibly being used as a mixture and under various salinity, pH and polarity conditions.

Separation, Detection and/or Characterization Process

The present invention also relates to a process for separating, detecting and/or characterizing at least one molecule of interest A comprising at least one step of using a kit as defined previously.

According to one embodiment, said molecule of interest may be present in a medium, and in particular in a complex medium.

The separation, detection and/or characterization process according to the invention may comprise at least one step of placing a compound of general formula (I) in contact with the molecule of interest A under conditions suitable for forming a bonding function L_{click} especially as described previously, followed by a step of placing the medium thus prepared in contact with a molecularly imprinted polymer under conditions suitable for the recognition of the bonding function L_{click}.

According to one embodiment, the medium before the placing in contact of the molecularly imprinted polymer may or may not be pretreated. The pretreatment may be an extraction, a concentration, a distillation, a dilution, or a change of pH, salinity or polarity conditions.

According to one embodiment, a compound of general formula (II) as defined previously may have been grafted onto the molecule of interest A before the reaction with the compound of general formula (I).

According to one embodiment, the process in accordance with the invention may also comprise a step of forming a purified, and optionally enriched, solution of molecule of interest.

This step may be performed, for example, by placing said molecularly imprinted polymer in contact with a medium that is suitable for breaking the interactions between said polymer and said bonding function L_{click}.

The molecularly imprinted polymer (MIP) in accordance with the invention allows extraction of the molecule(s) of interest, by molecular recognition of the bonding function L_{click}.

For the purposes of the invention, the expression “extraction of the molecule(s) of interest by molecular recognition of the bonding function L_{click}” means a step during which the interaction with the recognition sites of said MIP is sufficient to lead to the formation of a complex composed of the MIP bearing, in all or some of its recognition sites, at least the bonding function L_{click}.

The MIP may thus allow, according to one embodiment of the invention and after release of the extracted molecule(s) of interest, the production of a purified or even enriched solution of the molecule(s) of interest.

For the purposes of the invention, the expression “release of the extracted molecule(s) of interest” means a step during which the complex formed during the extraction of the molecule(s) of interest by molecular recognition of the bonding function L_{click} dissociates, for example following a modification of the pH, salinity, temperature, flow rate, pressure or solvent polarity conditions, leading to the presence of the molecule(s) of interest in a free form in solution.

According to another embodiment, the process in accordance with the invention may also include a step of qualitative, quantitative and/or semi-quantitative detection of the molecule of interest, for example via the detection of B.

According to one embodiment, B is a labeling species that is detectable, where appropriate, only after formation of the bonding function L_{click}.

According to another embodiment, the MIP may also emit a detectable signal, for example a variation in color or fluorescence, during the step of extraction of the molecule of interest.

### Applications

The separation, detection and/or characterization kit in accordance with the invention may be used for many applications.

In the context of the present invention, this term is also understood to denote a separation kit, a diagnostic kit, a screening kit, a kit for the analysis of chemical products, pollutants, toxic agents, medicaments, contaminants, drugs, perfumes, dyes or radiopharmaceuticals, and, for example, PET (positron emission tomography) radiotracers.

It may especially be a separation kit, a kit for the diagnosis or analysis of catalysts, reagents, products or by-products of a reaction, toxic agents, medicaments, contaminants, drugs, chemical products, perfumes, dyes, radiopharmaceuticals, for example PET (positron emission tomography) radiotracers, vitamins, proteins, amino acids and peptides, oligonucleotides, hormones, enzymes, DNA, peptide fragments, nucleoside fragments, nucleotide fragments, biomarkers, metabolites, chemical or biochemical warfare agents, sugars, polysaccharides, neurotransmitters, mycotoxins, pesticides, fungicides, herbicides, insecticides, fertilizers, antibodies, molecules indicating the safety and/or quality of foodstuffs, steroids or drugs.

The molecules of interest that it is possible, for example, to separate out, or even to analyze, by means of the kit according to the invention are especially reaction products, catalysts, reaction by-products, excess reagents or excess substrates.

Needless to say, the separation protocol will need to be adapted as a function of the intended objective, i.e., for example, to purify a molecule of interest or, on the other hand, to remove it from a given medium.

In particular, when the objective is to recover and purify, for example in order to recycle or characterize it, a given product, it may be useful to incorporate a cleavable bonding function onto the molecule of interest A optionally by means of the compound of general formula (II) in order to obtain, via an additional step, said product in its original form.

Thus, according to one embodiment of the invention, the kit may be intended for separating out tyrosine.

The examples given below are given as nonlimiting illustrations of the field of the invention.

### EXAMPLES

Examples 1 and 2 describe the synthesis of imprinted and nonimprinted materials No. 1 and demonstrate the selectivity of the imprinted material No. 1 toward triazole A and the nonselectivity toward azide and alkyne from which the triazole A is derived. This underlines the recognition of the unit L_{click}, i.e. the triazole unit, by the imprinted material No. 1.

Examples 3 and 4 describe the synthesis of a tyrosine derivative tagged with a unit L_{click} and its recognition by the imprinted material No, 1. The imprinted material No. 1 shows selectivity toward the tagged tyrosine noted triazole B, whereas tyrosine comprising an azide function (BocTyr(Azm)OMe) is not recognized. The imprinted material No. 1 shows selectivity toward a molecule (in this case tyrosine) tagged with a unit L_{click}.

Examples 5 and 6 describe the synthesis of imprinted and unimprinted materials No. 2 and their evaluation in solid-phase extraction. (SPE). In particular, Example 6 shows high selectivity of the imprinted material No. 2 toward the triazole C.

Example 7 describes the synthesis of triazoles D and E; triazole E being triphenylphosphine tagged with a unit L_{click} and triazole D being oxidized triazole E, also known as triphenylphosphine oxide tagged with a unit L_{click}.

Example 8 shows the selectivity of the imprinted material No. 2 toward triazole D by SPE.

Example 9 describes a direct application of use of the imprinted material No. 2 for the removal of a triphenylphosphine oxide derivative in the Mitsunobu reaction. Specially, during a Mitsunobu reaction (esterification in this example), triphenylphosphine oxide is a by-product that is difficult to remove from the reaction medium. The imprinted material No. 2 in accordance with the invention makes it possible to isolate the final product (ester) from the triphenylphosphine oxide via a simple SPE and by means of a preliminary click-chemistry reaction in situ in the reaction medium.

### Example 1

### Synthesis of the Imprinted and Unimprinted Materials No. 1 According to the Noncovalent Approach

### a) Synthesis of the template species N-[[1-(phenylmethyl)-[1,2,3]-triazol-4-yl]methyl]-benzamide

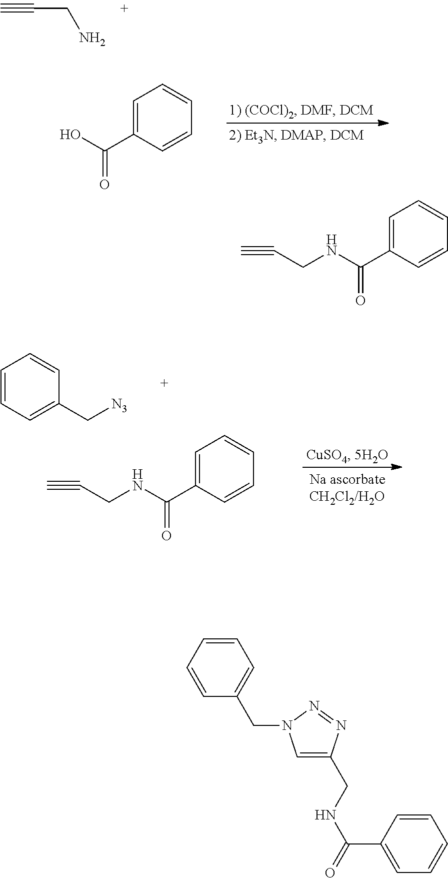

The reactions are performed under an inert atmosphere of nitrogen and are monitored by thin-layer chromatography (TLC). The commercial reagents are used as obtained. The reaction solvents are distilled as follows: tetrahydrofuran is distilled over sodium and benzophenone; triethylamine, dichloromethane, dimethylformamide and toluene were distilled over calcium hydride (CaH₂).

### Benzoyl Chloride

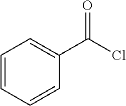

Benzoic acid (610 mg, 5 mmol) is dissolved in freshly distilled dichloromethane (10 mL) under nitrogen. Oxalyl chloride (635 μL, 7.5 mmol) is then added dropwise, followed by dimethylformamide (40 μL, 0.5 mmol). The reaction is stirred at room temperature for 20 hours. The medium is then concentrated on a rotary evaporator and then used directly in the reaction with propargylamine.

### N-Propargylbenzamide

According to Wipf P. et al., Org. Lett., 2004, 6 (20), 3593-3595

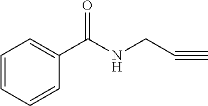

A solution of benzoyl chloride (703 mg, 5 mmol) in 2 mL of freshly distilled dichloromethane and triethylamine (836 μL, 6 mmol) are added to a solution of propargylamine (342 μL, 5 mmol) in 10 mL, of freshly distilled dichloromethane at 0° C. The mixture is stirred at 0° C. for 30 minutes and then for 3 hours at room temperature. The medium is poured into 50 mL of a 1M solution of hydrochloric acid in water. The aqueous phase is extracted with 2×25 mL of dichloromethane and the combined organic phases are then washed with saturated aqueous NaCl solution (2×25 mL) and dried over MgSO₄. After evaporating off the solvent on a rotary evaporator, the solid obtained is washed. The white solid (660 mg, 83%) is dried in a vacuum oven at 25° C.

¹H NMR (300 MHz CDCl₃, δ_{TMS}=0 ppm) δ 7.79 (D, 2H arom, J=7.5 Hz), 7.56-7.42 (m, 3H arom), 6.28 (br, 1H, NH), 4.26 (dd, 2H, J=5.1, 2.5 Hz, CH₂NH), 2.29 (t, 1H, J=2.4 Hz, C≡CH).

### N-[[1-(Phenylmethyl)-[1,2,3]-triazol-4-yl]methyl]-benzamide (triazole A)

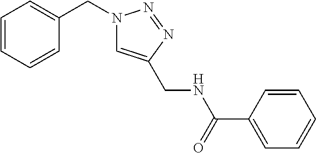

N-Propargylbenzamide (660 mg, 416 mmol) and benzyl azide (572 μL, 4.58 mmol) are dissolved in 4.6 mL of dichloromethane and 4.6 mL of distilled water. Copper sulfate pentahydrate (53 mg, 0.21 mmol) and sodium ascorbate (124 mg, 0.625 mmol) are then added. The medium is stirred at room temperature for 2 hours and then diluted with 30 mL of dichloromethane and 30 mL of distilled water. The phases are separated and the organic phase is dried over MgSO₄ and evaporated. The residue obtained is purified on a chromatography column (silica, eluent: 90/10 dichloromethane/methanol). A white solid is obtained (965 mg, 79%).

¹H NMR (300 MHz, DMSO, δ_{TMS}=0 ppm) δ 9.03 (t, 1H, J=4.5 Hz, NH), 8.03 (s, 1H, H triazole), 7.86 (d, 2H arom, J=6.8 Hz), 7.53-7.30 (m, 8H arom), 5.56 (s, 2H, CH₂ phenyl), 4.50 (d, 2H, J=5.7 Hz, CH₂NE).

### b) Synthesis of the Corresponding Imprinted and Unimprinted Materials No. 1

Ethylene glycol dimethacrylate is washed several times with a saturated basic NaCl solution to remove the inhibitor. It is dried over MgSO₄. The initiator azobisisobutyronitrile (AIBN) is recrystallized from acetone.

The imprinted material No. 1 is prepared by mixing 185 mg of N-[[1-(phenylmethyl)-[1,2,3]-triazol-4-yl]methyl]benzamide, 2.5 g of ethylene glycol dimethacrylate and 217 mg of methacrylic acid in 3.5 mL of anhydrous toluene. The mixture is degassed by bubbling nitrogen through for 10 minutes, and 33 mg of AIBN are then added. Polymerization is performed at 50° C. for 48 hours to form a white monolith.

The unimprinted material No. 1 is prepared by mixing 2.5 g of ethylene glycol dimethacrylate and 217 mg of methacrylic acid with 3.5 mL of anhydrous toluene. The mixture is degassed by bubbling nitrogen through for 10 minutes, and 33 mg of AIBN are then added. Polymerization is performed at 50° C. for 48 hours to form a white monolith.

The matrices prepared above are ground and then screened. The particles between 25 and 45 μm in size are introduced into a 250×2.1 mm HPLC column and then tamped by pressure and washed with a 5% mixture of acetic acid in acetonitrile/H₂O (97.5/2.5) and then with acetonitrile for the recognition study by HPLC, 2 HPLC columns are thus provided.

### Example 2

### Evaluation of the Recognition of Material No. 1

### a) By HPLC

Two solutions at 5 mM and 10 mM of triazole A in acetonitrile are injected onto the two columns filled, respectively, with imprint No. 1 and the unimprinted material No. 1.

The eluent used is acetonitrile at a flow rate of 1 mL/min. The detection of triazole A is performed with a UV detector. The injection volumes are 10 μL.

The k′ value (capacity factor) and the IF value (imprint factor) are determined to evaluate the recognition of triazole A on the matrices.

| Analyte | k′_{MIP No. 1} | k′_{unimprinted material No. 1} | IF |
|---|---|---|---|
| Triazole A 1 mM | 12.12 | 0.67 | 18 |
| Triazole A 5 mM | 5.54 | 0.56 | 9.9 |
| Triazole A 10 mM | 4.24 | 0.52 | 8.2 |
| Benzyl azide 1 mM | 0.12 | 0.11 | 1.09 |
| Benzyl azide 5 mM | 0.12 | 0.11 | 1.09 |
| N-Propargylbenzamide 5 mM | 0.22 | 0.13 | 1.69 |

HPLC conditions: eluent: ACN

Under the analytical conditions used, large recognition of imprint No. 1 for triazole A is observed. On the other hand, benzyl azide and N-propargylbenzamide are not at all retained by imprint No. 1. Large selectivity toward triazole A is obtained.

### b) By SPE

An SPE cartridge is prepared by introducing 50 mg of imprint No. 1 between two sinters. Prior to the extraction, 5 mL of dichloromethane are passed through the cartridge to condition it before introducing the solution to be percolated. Next, 1 mL of a solution containing 10 μg of N-[[1-(phenylmethyl)-[1,2,3]-triazol-4-yl]methyl]benzamide (triazole A), 2 μg of benzyl azide and 2 μg of N-propargylbenzamide in dichloromethane is percolated through the SPE cartridge. Several 1-mL fractions of dichloromethane are used as washing solution. Thereafter, the elution is performed with two 1-mL fractions of dichloromethane containing 1% acetic acid. The various fractions are then analyzed by UV-HPLC.

The table below gives the degrees of recovery (%) of N-[[1-(phenylmethyl)-[1,2,3]-triazol-4-yl]methyl]-benzamide (triazole A), of benzyl azide and of N-propargylbenzamide obtained during this extraction.

| | | | % degree of |
|---|---|---|---|
| | % degree of | % degree of | recovery of |
| | recovery of | recovery of | N-propargyl- |
| Fraction | triazole A | benzyl azide | benzamide |
| Percolation | 0 | 48 | 53 |
| Wash 1 | 0 | 35 | 25 |
| Wash 2 | 0 | 0 | 0 |
| Wash 3 | 0 | 0 | 0 |
| Wash 4 | 0 | 0 | 0 |
| Wash 5 | 0 | 0 | 0 |
| Elution 1 | 69 | 0 | 0 |
| Elution 2 | 19 | 0 | 0 |
| Total | 88 | 83 | 78 |

The imprinted material shows high selectivity toward triazole A and very low selectivity toward benzyl azide and N-propargylbenzamide.

### Example 3

### Production of N-[[1-(BocTyrOMe)-[1,2,3]-triazol-4-yl]methyl]benzamide (triazole B) by click-chemistry reaction between N-propargylbenzamide (a compound of general formula (I)) and a methyl ester of N-t-butyloxycarbonyl-O-azidomethyltyrosine (BocTyr(Azm)OMe)

According to Lee et al. Angew. Chem. Int. Ed., 2002, 42, No. 18, 3449-3451

### Methyl ester of N-t-butyloxycarbonyl-O-methylthiomethylyrosine (BocTyr(MTM)OMe)

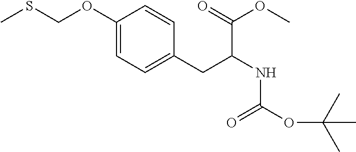

A solution of potassium t-butoxide (2.09 g, 18.64 mmol) in THF (20 mL) is added to a solution of methyl ester of N-t-butyloxycarbonyltyrosine (BocTyrOMe) (5 g, 16.95 mmol) and sodium iodide (255 mg, 1.7 mmol) in freshly distilled DMF (40 mL), cooled beforehand in an ice bath. Chloromethyl methyl sulfide (1.56 ml, 18.64 mmol) is added dropwise to the phenoxide thus formed (green solution). The reaction medium is gradually warmed to room temperature. After checking the disappearance of the starting material by TLC, the medium is diluted with ethyl acetate (80 mL) and washed with water (1×60 mL), a solution of citric acid in water (1×60 mL) and saturated aqueous NaCl solution (1×60 mL). The various aqueous phases are combined and extracted with ethyl acetate (2×80 mL). The combined organic phases are dried over MgSO₄ and evaporated on a rotary evaporator. The residue obtained is purified on a chromatography column (silica, eluent: 80/20 petroleum ether/ethyl acetate). 3.8 g of a clear liquid are obtained (yield=65%).

¹H NMR (300 MHz, CDCl₃, δ_{TMS}=0 ppm) δ 7.07 (AA¹BB¹, J=51 Hz, 8.5 Hz, 4H arom), 5.12 (s, 2H, CH₂S), 4.98 (br d, J=8.1 Hz, 1H, NH), 4.54 (q, J=6 Hz, 7.7 Hz, 1H, CH), 3.73 (s, 3H, OCH₃), 3.01 (m, 2H, CH₂), 2.25 (s, 3H, SCH₃), 1.42 (s, 9H, t-Bu) ppm.

### Methyl ester of N-t-butyloxycarboryl-O-azidomethyl-tyrosine (BocTyr(Azm)OMe)

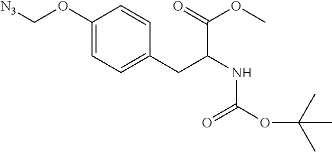

O,S-Acetal BocTyr(MTM)OMe (3.8 g, 10.7 mmol) is dissolved in dry dichloromethane (38 mL) and AT-chlorosuccinimide (1.57 g, 11.77 mmol) is added. The mixture is stirred at room temperature for 4 hours, followed by dropwise addition of trimethylsilyl chloride (1.5 mL, 11.77 mmol). After 6 hours of reaction, the medium is diluted with 30 mL of dichloromethane and 60 mL of saturated aqueous sodium hydrogen carbonate solution. The phases are separated and the aqueous phase is extracted with dichloromethane (2×60 mL). The organic phases are combined and concentrated under vacuum. The residue is then dissolved in 15 mL of dimethylformamide. A solution of sodium azide (1.04 g, 16.05 mmol) in 15 mL of distilled water is then added dropwise. The reaction is continued for 5 hours at room temperature. The medium is then diluted with 15 mL of saturated aqueous sodium hydrogen carbonate solution and washed with ethyl acetate (3×30 mL). The combined organic phases are dried over MgSO₄ and concentrated under vacuum; the residue is purified on a chromatography column (silica, eluent: 80/20 petroleum ether/ethyl acetate). A clear oil is obtained (1.6 g, 43%).

¹H NMR (300 MHz, CDCl₃, δ_{TMS}=0 ppm) δ 7.04-6.86 (AA¹BB¹, J=51 Hz, 8.5 Hz, 4H arom), 5.08 (s, 2H, CH₂N₃), 5.05 (br d, J=8.1 Hz, 1H, NH), 4.48 (q, J=6 Hz, 7.7 Hz, 1H, CH), 3.66 (s, 3H, OCH₃), 2.93 (m, 2H, CH₂), 1.1.36 (s, 9H, t-Bu) ppm.

### N-[[1-(BocTyrOMe)-[1,2,3]-triazol-4-yl]methyl]benzamide (triazole B)

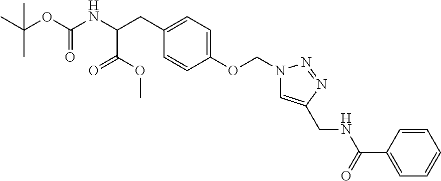

N-Propargylbenzamide (640 mg, 4.025 mmol) and BocTyr(Azm)OMe (1.46 g, 4.18 mmol) are dissolved in 4.5 mL of dichloromethane and 4.5 mL of distilled water. Copper sulfate pentahydrate (53 mg, 0.21 mmol) and sodium ascorbate (124 mg, 0.625 mmol) are then added. The medium is stirred at room temperature for 2 hours and then diluted with 30 mL of dichloromethane and 30 mL of distilled water. The phases are separated and the organic phase is dried over MgSO₄ and evaporated. The residue obtained is purified on a chromatography column (silica, eluent: 90/10 dichloromethane/methanol). A white solid is obtained (1.83 g, 90%).

¹H NMR (300 MHz, CDCl₃, δ_{TMS}=0 ppm) δ 7.8-7.11 (m, 6H, 5H arom+H triazole), 7.05-6.92 (AA¹B¹, J=51 Hz, 8.5 Hz, 4H arom), 6.17 (s, 2H, O—CH₂—N), 5.02 (br d, J=8.1 Hz, 1H, NH), 4.70 (d, 2H, J=5.7 Hz, CH₂NH), 4.51 (q, J=6 Hz, 7.7 Hz, 1H, CH), 3.69 (s, 3H, OCH₃), 2.93 (m, 2H, CH₂), 1.40 (s, 9H, t-Bu) porn.

### Example 4

### Evaluation of the Recognition of Triazole B by MIP No. 1

Two solutions at 5 mM and 10 mM of triazole A in acetonitrile, two solutions at 5 mM and 10 mM of triazole B in acetonitrile, and one solution at 5 mM of BocTyrAzm(OMe) in acetonitrile are injected onto the two columns filled, respectively, with imprint No. 1 and with the unimprinted material No. 1.

The eluent used is acetonitrile at a flow rate of 1 mL/min. The detection of triazole A, of triazole B and of BocTyrAzm(OMe) is performed with a UV detector. The injection volumes are 10 μL.

The retention time (tr), k′ (capacity factor) and IF (imprint factor) values are determined for each of these compounds, on each of these columns.

The results are as follows.

| Analyte | k′_{MIP No. 1} | k′_{unimprinted material No. 1} | IF |
|---|---|---|---|
| Triazole A 1 mM | 12.22 | 0.71 | 17.2 |
| Triazole A 5 mM | 6.29 | 0.65 | 9.7 |
| Triazole B 5 mM | 1.23 | 0.21 | 5.86 |
| Triazole B 1 mM | 1.69 | 0.23 | 7.35 |
| BocTyrAzm(OMe) 5 mM | 0.08 | 0.06 | 1.33 |

HPLC conditions: eluent: ACN

Under the analytical conditions used, greater recognition of imprint No. 1 for triazole B than for the corresponding azide noted BocTyrAzm(OMe) is observed.

MIP No. 1 in accordance with the invention thus makes it possible to selectively separate the triazole B containing a bonding function L_{ciick} of triazole type.

Triazole A serves herein as a reference.

### Example 5

### Synthesis of the Imprinted and Unimprinted Materials No. 2

### a) Synthesis of the Template Species N-[[1-(3,5-bistrifluoromethylbenzyl)-5H-1,2,3-triazol-4-yl]methyl]benzamide

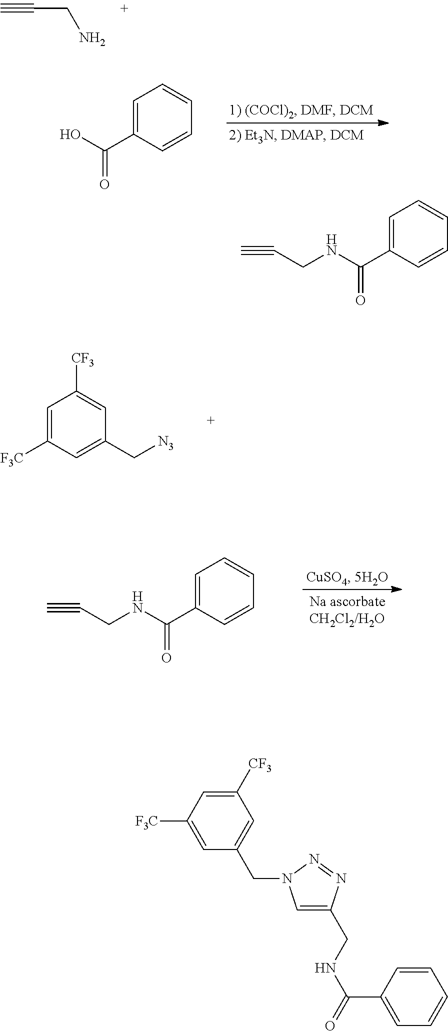

The reactions are conducted under an inert atmosphere of nitrogen and monitored by thin-layer chromatography (TLC). The commercial reagents are used as obtained. The reaction solvents are distilled as follows: tetrahydrofuran is distilled over sodium and benzophenone; triethylamine, dichloromethane and dimethylformamide were distilled over calcium hydride (CaH₂) and chloroform was distilled over phosphorus pentoxide (P₂O₅).

### LC-MS Conditions:

Hypersil gold, 50×2.1 mm column, gradient: 0.1% HCOOH in water for 2 minutes and then 80/20 ACN/0.1% HCOOH water up to 18 minutes and then constant up to 30 minutes. 0.2 mL/min, λ=230 nm.

### Benzoyl Chloride

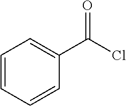

Benzoic acid (610 mg, 5 mmol) is dissolved in freshly distilled dichloromethane (10 mL) under nitrogen. Oxalyl chloride (635 μL, 7.5 mmol) is then added dropwise, followed by dimethylformamide (40 μL, 0.5 mmol). The reaction is stirred at room temperature for 20 hours. The medium is then concentrated on a rotary evaporator and then used directly in the reaction with propargylamine.

### N-Propargylbanzamide

According to Wipf P. et al., Org. Lett., 2004, 6 (20), 3593-3595

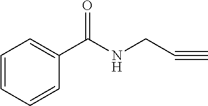

A solution of benzoyl chloride (703 mg, 5 mmol) in 2 mL of dichloromethane and triethylamine (836 μL, 6 mmol) are added to a solution of propargylamine (342 μL, 5 mmol) in 10 mL of dichloromethane at 0° C. The mixture is stirred at 0° C. for 30 minutes and then for 3 hours at room temperature. The medium is poured into 50 mL of a 1M solution of hydrochloric acid in water. The aqueous phase is extracted with 2×25 mL, of dichloromethane and the combined organic phases are then washed with saturated aqueous NaCl solution (2×25 mL) and dried over magnesium sulfate. After evaporating off the solvent on a rotary evaporator, the solid obtained is washed. The white solid (660 mg, 83%) is dried in a vacuum oven at 25° C.

¹H NMR (300 MHz CDCl₃, δ_{TMS}=0 ppm) δ 7.79 (d, 2H arom, J=7.5 Hz), 7.56-7.42 (m, 3H arom), 6.28 (br, 1H, NH), 4.26 (dd, 2H, J=5.1, 2.5 Hz, CH₂NH), 2.29 (t, 1H, J=2.4 Hz, C≡CH).

### 3,5-Bistrifluoromethylbenzyl azide

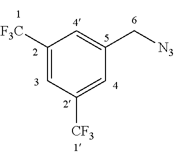

3,5-Bistrifluoromethyl chloride (3.93 g, 15 mmol) is dissolved in a 4/1 acetone/distilled water mixture (80 mL). Sodium azide (5.85 g, 90 mmol) is added and the medium is refluxed for 16 hours. The medium is cooled to 20° C. and evaporated on a rotary evaporator. The residue is taken up in 50 mL of dichloromethane and the organic phase is then washed with water (3×25 mL) and with saturated aqueous NaCl solution (2×25 mL). The organic phase is dried over magnesium sulfate and evaporated. A colorless liquid, is obtained (3.63 g, 90%).

¹H NMR (300 MHz CDCl₃, δ_{TMS}=0 ppm) δ 7.88 (s, 1H₃ arom), 7.81 (s, 2H arom, H₄, H₄′), 4.56 (s, 2H, CH₂N₃).

¹⁹F NMR (282 MHz, CDCl₃, δ_{CFCl3}=0 ppm) δ −63 ppm.

¹³C NMR (75 MHz CDCl₃, δ_{TMS}=0 ppm) δ 138.5 (C₅), 132.2 (q, ²J_{C-F}=34 Hz, C₄, C₄′) 128 (C₂, C_{2′),} 123.3 (q, ¹J_{C-F}=272 Hz, C₁), 122.3 (q, ³J_{C-F}=3.8 Hz, C₃), 53.6 (C₆, CH₂N₃).

GC-MS: 98.5% purity, m/z=269.

### N-[[1-(3,5-Bistrifluoromethylbenzyl)-5H-1,2,3-triazol-4-yl]methyl]benzamide (triazole C)

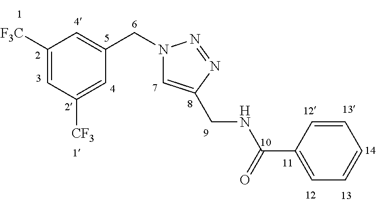

N-Propargylbenzamide (1.4 g, 8.78 mmol) and 3,5-bistrifluoromethylbenzyl azide (2.6 g, 9.66 mmol) are dissolved in 105 ml., of dichloromethane and 105 mL, of distilled water. Copper sulfate pentahydrate (110 mg, 0.44 mmol) and sodium ascorbate (261 mg, 1.32 mmol) are then added. The medium is stirred at room temperature for 16 hours. The phases are separated and the organic phase is dried over magnesium sulfate and evaporated. The residue obtained is purified on a chromatography column (silica, eluent: 90/10 dichloromethane/methanol). A beige-colored solid is obtained (3.71 g, 98%).

¹H NMR (300 MHz, CDCl₃, δ_{TMS}=0 ppm) δ 7.9 (s, 1H, H₆ triazole), 7.75 (d, 2H arom, J=7.5 Hz), 7.71 (m, 3H arom), 7.5 (m, 3 CH ar), 7.10 (t, 1H, J=Hz, NH), 5.63 (s, 2H, CH₂ phenyl), 4.73 (dd, 2H, J=5.1, 2.5 Hz, CH₂NH).

¹⁹F NMR (282 MHz, CDCl₃, δ_{CFCl3}=0 ppm) δ −63 ppm.

¹³C NMR (75 MHz, CDCl₃, δ_{TMS}=0 ppm) δ 167.8 (C₁₀, C═O), 144.2 (C₈), 137.2 (C₅), 133.8 (C₁₁), 132.6 (C₂, C_{2′}), 131.8 (C₁₄), 128.6 (q, ²J_{C-F}=34 Hz, C₄, C_{4′}), 128.3 (C₁₃), 127.3 (C₁₂), 123.3 (q, ¹J_{C-F}=272 Hz, C₁), 122.9 (q, ³J_{C-F}=3.8 Hz, C₃), 114.0 (C₇), 53 (C₆), 35.3 (C₉).

HPLC-MS conditions: triazole C: tr=18.97 min

MS (ESI⁺): m/z=428.99 [M+H]⁺ 428.34 calculated.

### b) Synthesis of the Corresponding Imprinted and Unimprinted Materials No. 2

Trimethylpropane trimethacrylate is washed several times with saturated basic NaCl solution to remove the inhibitor. It is dried over MgSO₄. The initiator azobisisobutyronitrile (AIBN) is recrystallized from acetone.

The imprinted material No. 2 is prepared by mixing 1.9 g of N-[[1-(3,5-bistrifluoromethylbenzyl)-1H-1,2,3-triazol-4-yl]methyl]benzamide (triazole C), 5 g of trimethylpropane trimethacrylate and 382 mg of methacrylic acid in 5.3 mL of anhydrous chloroform. The mixture is degassed by bubbling nitrogen through for 10 minutes, and is then placed at 70° C. for 15 minutes to obtain complete dissolution. 39 mg of AIBN are then added. Polymerization is performed at 50° C. for 48 hours to form a colored monolith.

The unimprinted material No. 2 is prepared by mixing 5 g of trimethylpropane trimethacrylate and 217 mg of methacrylic acid in 5.3 mL of anhydrous chloroform. The mixture is degassed by bubbling nitrogen through for 10 minutes, and 39 mg of AIBN are then added. Polymerization is performed at 50° C. for 48 hours to form a colored monolith.

The matrices prepared above are coarsely ground and then washed by ASE (Automated Solvent Extraction) with a mixture of 5% acetic acid in methanol/H₂O (97.5/2.5) and then methanol. The matrices are then finely ground and then screened. The particles between 25 and 45 μm in size are washed by ASE in the same manner as previously for the SPE recognition study.

### Example 6

### Evaluation of the Recognition of Material No. 2 by SPE

An SPE cartridge is prepared by introducing 50 mg of imprint No. 2 between two sinters. Prior to the extraction, 5 mL of dichloromethane are passed through the cartridge to condition it, before introducing the solution to be percolated. Next, 1 mL of a solution containing 44 μg of N-[[1-(3,5-bistrifluoromethylbenzyl)-5H-1,2,3-triazol-4-yl]methyl]benzamide (triazole C) in dichloromethane is percolated through the SPE cartridge. Several 1-mL fractions of dichloromethane are used as washing solution. Thereafter, the elution is performed with two 1-mL fractions of dichloromethane containing 2.5% acetic acid. The various fractions are then analyzed by UV-HPLC.

The table below gives the degrees of recovery (%) of N-[[1-(3,5-bistrifluoromethylbenzyl)-5H-1,2,3-triazol-4-yl]methyl]benzamide (triazole C) obtained during this extraction.

| | % degree of recovery |
|---|---|
| Fraction | of triazole C |
| Percolation | 0 |
| Wash 1 | 0 |
| Wash 2 | 0 |
| Wash 3 | 0 |
| Wash 4 | 1 |
| Wash 5 | 1 |
| Elution 1 | 83 |
| Elution 2 | 7 |
| Total | 92 |

The imprinted material shows high selectivity toward triazole C.

### Example 7

### Production of N-[[1-(3,5-bistrifluoromethylbenzyl)-5H-1,2,3-triazol-4-yl]methyl]diphenylphosphinobenzamide oxide by click-chemistry reaction between N-propargyldiphenylphosphinobenzamide and 3,5-bistrifluoromethylbenzyl azide

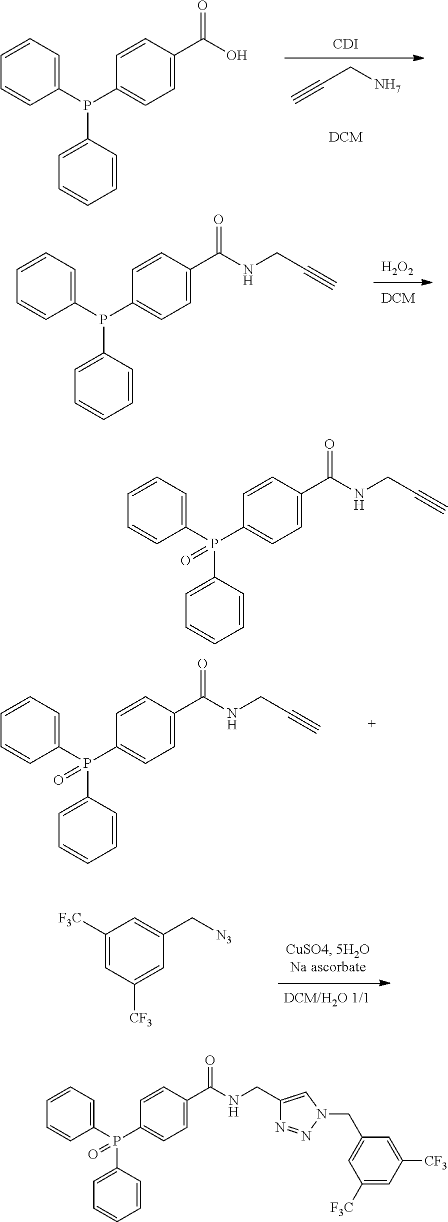

### N-Propargyldiphenylphosphinobenzamide

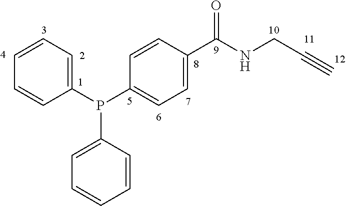

Diphenylphosphinobenzoic acid (1 g, 3.27 mmol) is dissolved in 100 ml, of dichloromethane and carbonyldiimidazole (795 mg, 4.91 mmol) is then added. After stirring for 2 hours at room temperature, propargylamine (336 μL, 4.91 mmol) is added to the reaction medium and the reaction is stirred at room temperature for 16 hours. The medium is poured into 50 mL of aqueous 1N hydrochloric acid solution and the aqueous phase is then extracted with 2×50 mL of dichloromethane. The combined organic phases are washed with saturated aqueous NaCl solution (2×50 mL), dried over magnesium sulfate and evaporated. The residue obtained is purified on a chromatography column (silica, eluent: 80/20 dichloromethane/ethyl acetate). A slightly yellow foam is obtained (955 mg, 76%).

¹H NMR (300 MHz, CDCl₃, δ_{TMS}=0 ppm) δ 7.73 (d, 2H arom, J=7.2 Hz, H₇), 7.36 (m, 12H arom, H₂, H₃, H₄, H₆), 6.82 (t, 1H, J=5.0 Hz, NH), 4.24 (dd, 2H, J=5.3, 2.5 Hz, H₁₀), 2.24 (t, 1H, J=2.5 Hz, H₁₂).

³¹P NMR (121 MHz, CDCl₃, δ_{H3PO4}=0 ppm) δ −4.96 ppm.

¹³C NMR (75 MHz CDCl₃, δ_{TMS}=0 ppm), δ 166.8 (C₉, C═O), 142.7 (d, ¹J_{C-P}=13.6 Hz, C₅), 136.2 (d, ¹J_{C-P}=10.4 Hz, C₁), 133.8 (d, ²J_{C-P}=19.7 Hz, C₂), 133.7 (d, ²J_{C-P}=19.7 Hz, C₆), 133.6 (C₈), 129.1 (C₄), 128.7 (d, ³J_{C-P}=7.1 Hz, C₃), 126.9 (d, ³J_{C-P}=7.1 Hz, C₇), 79.3 (C₁₁), 72.0 (C₁₂), 29.8 (C₁₀).

HPLC-MS conditions: N-propargyldiphenylphosphinobenzamide: tr=20.71 min

MS (ESI⁺): m/z=344.68 [M+H]⁺ 4 Hr 344.37 calculated.

### N-Propargyldiphenylphosphinobenzamide oxide

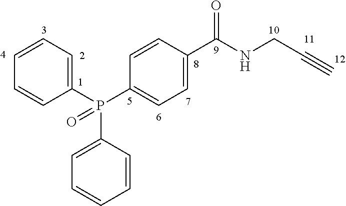

N-Propargyldiphenylphosphinobenzamide (3.33 g, 9.708 mmol) is dissolved in 40 mL of dichloromethane and the medium is then cooled to 0° C. 35% aqueous hydrogen peroxide solution (3.77 mL, 38.83 mmol) is then added. After reaction for 1 hour, 40 mL of water are added. The phases are separated and the organic phase is washed with saturated sodium bicarbonate solution. After drying over MgSO₄, the organic phase is evaporated. A white foam is obtained (2.97 g, 85%).

¹H NMR (300 MHz, CDCl₃, δ_{TMS}=0 ppm) δ 8.26 (t, 1H, J=5 Hz, NH), 7.84 (dd, 2H arom, J=2.5, 8.3 Hz, H₇), 7.46 (m, 12H arom, H₂, H₃, H₄, H₆), 4.2 (dd, 2H, J=2.5, 5.3 Hz, H₁₀), 2.17 (t, 1H, J=2.5 Hz, H₁₂).

³¹P NMR (121 MHz, CDCl₃, δ_{H3PO4}=0 ppm) δ 29.3 ppm.

¹³C NMR (75 MHz, CDCl₃, δ_{TMS}=0 ppm) δ 166.8 (C₉, C═O), 137.8 (d, ¹J_{C-P}=2.8 Hz, C₅), 135.4 (d, ¹J_{C-P}=102.6 Hz, C₁), 133.8 (d, ⁴J_{C-P}=2.8 Hz, C₄), 132.2 (d, ²J_{C-P}=10.4 Hz, C₆), 131.1 (d, ²J_{C-P}=10 Hz, C₂) 130.9 (C₈), 128.7 (d, ³J_{C-P}=12.1 Hz, C₃), 127.6 (d, ³J_{C-P}=12.1 Hz, C₇), 79.3 (C₁₁), 72.0 (C₁₂), 29.8 (C₁₀).

HPLC-MS conditions: N-propargyldiphenylphosphinobenzamide oxide: tr=15.9 min

MS (ESI⁺): m/z=360.16 [M+H]⁺ 360.37 calculated.

### N-[[1-(3,5-Bistrifluoromethylbenzyl)-5H-1,2,3-triazol-4-yl]methyl]diphenylphosphorylbenzamide oxide (triazole D)

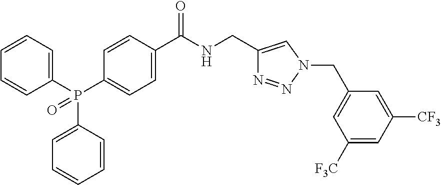

N-Propargyldiphenylphosphinobenzamide oxide (350 mg, 1.026 mmol) and 3,5-bistrifluoromethylbenzyl azide (303 mg, 1.128 mmol) are dissolved in 15 mL of dichloromethane and 15 mL of distilled water. Copper sulfate pentahydrate (13 mg, 0.051 mmol) and sodium ascorbate (30.5 mg, 0.154 mmol) are then added. The medium is stirred at room temperature for 16 hours. The phases are separated and the organic phase is dried over magnesium sulfate and evaporated. The residue obtained is purified on a chromatography column (silica, eluent: 2/8 dichloromethane/ethyl acetate and then 90/10 ethyl acetate/methanol). A beige-colored solid is obtained (471 mg, 73%).

¹H NMR (300 MHz, CDCl₃, δ_{TMS}=0 ppm) δ 8.72 (t, 1H, J=5 Hz, NH), 7.88 (s, 1H, H17), 7.81 (s, 2H, H15), 7.73 (d, 2H arom, J=7.2 Hz, H₇), 7.69 (s, 1H, H12), 7.36 (m, 12H arom, H2, H₃, H₄, H₆), 5.56 (s, 2H, H13), 4.63 (d, 2H, J=4.9 Hz, H10).

¹⁹F NMR (282 MHz, CDCl₃, δ_{CFCl3}=0 ppm) δ −62.9 ppm.

³¹P NMR (121 MHz, CDCl₃, δ_{H3PO4}=0 ppm) δ 29 ppm.

¹³C NMR (75 MHz, CDCl₃, δ_{TMS}=0 ppm) δ 167.1 (C₉, C═O), 137.6 (d, ¹J_{C-P}=2.8 Hz, C₅), 137.2 (C₁₄), 135.9 (d, ¹J_{C-P}=102.1 Hz, C₁), 133.3 (C₁₁), 132.9 (C₁₆), 132.4 (d, ⁴J_{C-P}=2.8 Hz, C₄), 132.2 (d, ²J_{C-P}=10.4 Hz, C₆), 132.0 (d, ²J_{C-P}=10 Hz, C₂), 131.1 (C₈), 128.8 (d, ³J_{C-P}=12.5 Hz, C₃), 128.2 (d, ²J_{C-P}=34 Hz, C₁₅), 127.6 (d, ³J_{C-P}=12.1 Hz, C₇), 123.1 (C₁₂), 123.0 (q, ¹J_{C-P}=272 Hz, C₁₀), 122.9 (q, ²J_{C-F}==3.8 Hz, C₁₇), 52.9 (C₁₃), 35.6 (C₁₀). HPLC-MS conditions: triazole D: tr=23.15 min

MS (ESI⁺): m/z=629.61 [M+H]⁺ 628.51 calculated.

### N-[[1-(3,5-Bistrifluoromethylbenzyl)-5H-1,2,3-triazol-4-yl]methyl]diphenylphosphorylbenzamide (triazole E)

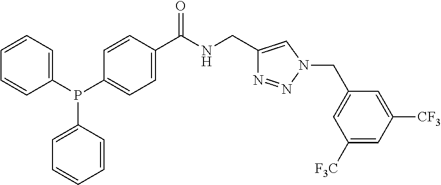

N-[[1-(3,5-Bistrifluoromethylbenzyl)-5H-1,2,3-triazol-4-yl]methyl]diphenylphosphorylbenzamide oxide (50 mg, 0.08 mmol) (triazole D) is dissolved in 3 mL of dry toluene under nitrogen. Triethylamine (18 μL, 0.128 mmol) and trichlorosilane (13 μL, 0.128 mmol) are added and the medium is refluxed for 4 hours. After cooling to room temperature, 4 mL of 20% sodium hydroxide in water are added. The phases are separated and the organic phase is then washed with saturated aqueous NaCl solution, dried over MgSO₄ and then evaporated. A white solid is obtained (38 mg, 78%).

HPLC-MS conditions: triazole E: tr=19.40 min

MS (ESI⁺): m/z=612.91 [M+H]⁺ 612.51 calculated.

### Example 8

### Evaluation of the Recognition of Triazole D by the Imprinted Material No. 2

An SPE cartridge is prepared by introducing 50 mg of imprint No. 2 between two sinters. Prior to the extraction, 5 ml of dichloromethane are passed through the cartridge to condition it, before introducing the solution to be percolated. Next, 1 mL of a solution containing 4 μg of N-[[1-(3,5-bistrifluoromethylbenzyl)-5H-1,2,3-triazol-4-yl]methyl]diphenylphosphinobenzamide oxide (triazole D) in dichloromethane is percolated through the SPE cartridge. Several 1-mL fractions of dichloromethane are used as washing solution. Thereafter, the elution is performed with two 1-mL fractions of dichloromethane containing 2.5% acetic acid. The various fractions are then analyzed by UV-HPLC.

The table below gives the degrees of recovery (%) of N-[[1-(3,5-bistrifluoromethylbenzyl)-5H-1,2,3-triazol-4-yl]methyl]diphenylphosphinobenzamide oxide (triazole D) obtained during this extraction.

| | % degree of recovery |
|---|---|
| Fraction | of triazole D |
| Percolation | 0 |
| Wash 1 | 0 |
| Wash 2 | 0 |
| Wash 3 | 0 |
| Wash 4 | 0 |
| Wash 5 | 0 |
| Elution 1 | 92 |
| Elution 2 | 2 |
| Total | 94 |

The imprinted material No. 2 shows high selectivity toward triazole D.

### Example 9

### Application of Material No. 2 in Organic Synthesis—Mitsunobu Reaction and Separation of a Reaction by-Product by Using the Process in Accordance with the Invention

### a) Mitsunobu Reaction: Synthesis of Ethyl 3,5-Dinitrobenzoate and Formation of an In-Situ L_{click} Unit in the Reaction Medium

### Ethyl 3,5-dinitrobenzoate

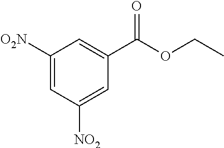

3,5-Dinitrobenzoic acid (62 mg, 0.292 mmol) and diisopropyl azodicarboxylate (58 μL, 0.292 mmol) are dissolved in 1 mL of diethyl ether. A solution of N-propargyldiphenylphosphinobenzamide (100 mg, 0.292 mmol) and ethanol (34 μL, 0.584 mmol) in 1 mL of diethyl ether is added to the preceding solution and the medium is stirred at room temperature until consumption of the 3,5-dinitrobenzoic acid is complete. The diethyl ether is then evaporated off by flushing with nitrogen, and the medium is taken up in 5 mL of a 1/1 mixture of dichloromethane and water. 3,5-Bistrifluoromethylbenzyl azide (95 mg, 0.350 mmol) is introduced into the medium, followed by copper sulfate pentahydrate (7.3 mg, 0.029 mmol) and sodium ascorbate (17.4 mg, 0.088 mmol). The reaction is then stirred for 18 hours at room temperature. At the end of the reaction, the medium is diluted with 10 mL of a 1/1 mixture of dichloromethane and water. The phases are separated and the organic phase is then washed with saturated aqueous NaHCO₃ solution and then with saturated aqueous NaCl solution, dried over magnesium sulfate and evaporated. The crude product obtained is then purified on an SPE cartridge.

### b) Purification by SPE

An SPE cartridge is prepared by introducing 50 mg of imprint No. 2 between two sinters. Prior to the extraction, 5 mL of dichloromethane are passed through the cartridge to condition it, before introducing the solution to be percolated. Next, 1 mL of a solution containing the preceding reaction mixture in dichloromethane is percolated through the SPE cartridge. Several 1-mL fractions of dichloromethane are used as washing solution. Thereafter, the elution is performed with two 1-mL fractions of dichloromethane containing 2.5% acetic acid. The various fractions are then analyzed by UV-HPLC.

The table below gives the degrees of recovery (%) of ethyl 3,5-dinitrobenzoate and of N-[(1-(3,5-bistrifluoromethylbenzyl)-5H-1,2,3-triazol-4-yl)methyl]diphenylphosphinobenzamide oxide (triazole D) obtained during this extraction.

| | % degree of | |
|---|---|---|
| | recovery of | % degree of |
| Fraction | triazole D | recovery of ester |
| Percolation | 0 | 82 |
| Wash 1 | 1 | 15 |
| Wash 2 | 1 | 0 |
| Wash 3 | 1 | 0 |
| Wash 4 | 1 | 0 |
| Wash 5 | 1 | 0 |
| Elution 1 | 99 | 0 |
| Elution 2 | 1 | 0 |
| Total | 105 | 97 |

Percolation of the reaction medium through the imprinted material No. 2 allows isolation of the ester, and the triazole D comes out only on elution. The ester is thus isolated very easily by SPE on the imprinted material No. 2 via washes with dichloromethane.

Formation of the L_{click} unit was performed in situ in the reaction medium in a quantitative yield, allowing the separation of the tagged triphenylphosphine oxide from the ester, by means of using the imprinted material No. 2 in accordance with the invention.

1.-20. (canceled)

21. A kit that is useful for the separation, detection and/or characterization of at least one molecule of interest A, said kit comprising at least:
(i) one compound of general formula (I) below:
B—(R)ₙ—Z (I)
in which:
B represents a hydrogen atom or a detectable labeling species;
R represents a C₁-C_{10,000} hydrocarbon-based unit that may be polymeric or nonpolymeric;
n represents 0 or 1, with n being equal to 1 when B represents a hydrogen atom;
Z represents a functional group that is capable of reacting in a click-chemistry reaction, to form a bonding function L_{click}, and
(ii) a molecularly imprinted polymer intended for the molecular recognition of at least said bonding function L_{click}.

22. A kit as claimed in claim 21, wherein R incorporates one or more heteroatoms selected from N, O, S, Br, Cl, F, P, B, Si and/or one or more metals.

23. The kit as claimed in claim 21, in which said molecularly imprinted polymer is configured for the molecular recognition of a unit R-L_{click}, B-L_{click} or B—R-L_{click}.

24. The kit as claimed in claim 21, said click-chemistry reaction being a 1,3-dipolar cycloaddition reaction or a reaction of Diels-Adler type.

25. The kit as claimed in claim 21, in which Z is selected from azides, alkynes, nitriles, dienes, and alkenes.

26. The kit as claimed claim 21, wherein B is a labeling species that is detectable only after the formation of the bonding function L_{click}.

27. The kit as claimed in claim 26, wherein B is a labeling species that is detectable by fluorescence.

28. The kit as claimed in claim 21, further comprising at least one compound of general formula (II) below:
X—R′—Y (II)
in which:
X represents a functional group that is reactive toward at least one functional group W borne by said molecule of interest A;
R′ represents a C₁-C_{10,000} hydrocarbon-based unit that may be polymeric or nonpolymeric; and
Y represents the functional group that is reactive in a click-chemistry reaction toward the functional group Z, to form the bonding function L_{click}.

29. The kit as claimed in claim 28, wherein R′ incorporates one or more heteroatoms selected from N, O, S, Br, Cl, F, P, B, Si, and/or one or more metals.

30. The kit as claimed in claim 28, in which said molecularly imprinted polymer is configured for the molecular recognition of a unit R-L_{click}-R′, B-L_{click}-R′, or B—R-L_{click}-R′.

31. The kit as claimed in claim 28, wherein the functional group X is capable of forming a bonding function.

32. The kit as claimed in claim 31, wherein said bonding function is cleavable by reaction with said functional group W.

33. The kit as claimed in claim 28, in which X is selected from carboxylic acid, amine, hydroxyl, hydroxylamine, nitrile, thiol, anhydride, acid halide, and iso(thio)cyanate functions.

34. The kit as claimed in claim 28, comprising at least two compounds of general formula (II), said compounds comprising, respectively, identical functional groups Y and functional groups X of different nature.

35. The kit as claimed in any claim 21, in which the molecularly imprinted polymer is configured to be used on an extraction column.

36. The kit as claimed in claim 21, further comprising a device for semiquantitative analysis and/or a device for quantitative analysis of the molecule of interest, based on the detection of the species B.

37. The kit as claimed in claim 21, which is configured for the separation of a molecule of interest present in a complex medium.

38. A process for separating, detecting and/or characterizing at least one molecule of interest A that may be present in a medium, the method comprising at least one step of using a kit comprising at least:
(i) one compound of general formula (I) below:
B—(R)ₙ—Z (I)
in which:
B represents a hydrogen atom or a detectable labeling species;
R represents a C₁-C_{10 000} hydrocarbon-based unit that may be polymeric or nonpolymeric;
n represents 0 or 1, with n being equal to 1 when B represents a hydrogen atom; and
Z represents a functional group that is capable of reacting in a click-chemistry reaction to form a bonding function L_{click}, and
(ii) a molecularly imprinted polymer intended for the molecular recognition of at least said bonding function L_{click}.

39. The process as claimed in claim 38 wherein the medium is a complex medium.

40. The process as claimed in claim 38, further comprising at least one step of placing a compound of general formula (I) and the molecule of interest A in contact under conditions suitable for forming a bonding function L_{click} followed by a step of placing the medium thus prepared in contact with a molecularly imprinted polymer under conditions suitable for recognizing the bonding function L_{click}.

41. The process as claimed in claim 40, wherein a compound of general formula (II):
X—R′—Y (II)
in which:
X represents a functional group that is reactive toward at least one functional group W borne by said molecule of interest A;
R′ represents a C₁-C_{10,000} hydrocarbon-based unit that may be polymeric or nonpolymeric; and
Y represents the functional group that is reactive in a click-chemistry reaction toward the functional group Z, to form the bonding function L_{click};
is grafted onto the molecule of interest A before the reaction with the compound of general formula (I).

42. The process as claimed in claim 38, further comprising a step of forming a purified solution of molecule of interest.

43. The process as claimed in claim 42, wherein said solution is enriched of molecule of interest.

44. The process as claimed in claim 42, said step being performed by placing said molecularly imprinted polymer in contact with a medium that is suitable for breaking interactions between said polymer and said bonding function L_{click}.

45. The process as claimed in claim 38, further comprising a step of qualitative, quantitative, and/or semiquantitative detection of the molecule of interest.

46. The process as claimed in claim 45, wherein said step of detection of the molecule of interest is via the detection of B.

47. A method for extraction, detection, separation, purification, absorption, adsorption, retention or controlled release, qualification and/or quantification of biomarkers in biology, using a kit comprising at least:
(i) one compound of general formula (I) below:
B—(R)ₙ—Z (I)
in which:
B represents a hydrogen atom or a detectable labeling species;
R represents a C₁-C_{10 000} hydrocarbon-based unit that may be polymeric or nonpolymeric;
n represents 0 or 1, with n being equal to 1 when B represents a hydrogen atom; and
Z represents a functional group that is capable of reacting in a click-chemistry reaction, to form a bonding function L_{click}, and
(ii) a molecularly imprinted polymer intended for the molecular recognition of at least said bonding function L_{click}.

48. The method as defined in claim 47, for using in proteomics, metabolomics or genomics or in applications selected from sensors, parallel synthesis, catalysis of chemical reactions, radiopharmacy, screening of molecules, directed chemical synthesis, sample treatment, combinatorial chemistry, chiral separation, group protection, equilibrium shifting, medicaments using polymers, and encapsulation.
